(19) United States
(12) Patent Application Publication
(10) Pub. No.: US 2023/0240889 A1
(43) Pub. Date: Aug. 3, 2023

(54) EQUALIZING MULTI-CHANNEL DRIVING SIGNALS OF SEGMENTED PIEZOELECTRIC CRYSTALS

(71) Applicant: JOHNSON & JOHNSON SURGICAL VISION, INC., Irvine, CA (US)

(72) Inventors: Amit Fuchs, Hogla (IL); Assaf Govari, Haifa (IL); Eran Aharon, Haifa (IL)

(21) Appl. No.: 17/590,184

(22) Filed: Feb. 1, 2022

(51) Int. Cl. A61F 9/007; B06B 1/02

(52) U.S. Cl. CPC A61F 9/00745; B06B 1/0276; B06B 2201/55; B06B 2201/76

(57) ABSTRACT

A system and method for driving a medical probe. The system including a processor configured to apply respective harmonic electrical signals to two or more piezoelectric crystals coupled with a tip of the medical probe so as to cause the tip to vibrate; and a balancer, which is configured to iteratively adjust the signals to equalize a selected parameter of the signals, so as to cause the tip to vibrate at a predefined trajectory. The method including applying respective harmonic electrical signals to two or more piezoelectric crystals coupled with a tip of the medical probe so as to cause the tip to vibrate. The signals are iteratively adjusted to equalize a selected parameter of the signals, so as to cause the tip to vibrate at a predefined trajectory.

### FIELD OF THE DISCLOSURE

The present disclosure relates generally to piezoelectric-vibration-based medical devices, and particularly to phacoemulsification systems.

### BACKGROUND

A cataract is a clouding and hardening of the eye's natural lens, a structure which is positioned behind the cornea, iris and pupil. The lens is made up mostly of water and protein, and as people age these proteins change and may begin to clump together, obscuring portions of the lens. Phacoemulsification cataract surgery can be used to correct this condition. In this procedure, a surgeon makes a small incision in the sclera or cornea of the eye. Then a portion of the anterior surface of the lens capsule is removed to gain access to the cataract. The surgeon then inserts the tip of a phacoemulsification probe into the lens capsule. The tip vibrates at ultrasonic frequency to sculpt and emulsify the cataract while a pump aspirates particles and fluid from the eye through the tip. Aspirated fluids are replaced with irrigation of a balanced salt solution to maintain the anterior chamber of the eye. After removing the cataract with phacoemulsification, the softer outer lens cortex is removed with suction. An intraocular lens (IOL) is then introduced into the empty lens capsule to restore the patient's vision.

For safe, efficient phacoemulsification, it is important that the vibration of the tip of the probe be precisely controlled. Various techniques to vibrate a phacoemulsification needle of a probe in a controlled manner were proposed in the patent literature.

The present disclosure will be more fully understood from the following detailed description of the examples thereof, taken together with the drawings in which:

### BRIEF DESCRIPTION OF THE DRAWINGS

FIG. 1 is a schematic, pictorial view, along with a side view, of a phacoemulsification system, in accordance with an example of the present disclosure;

FIG. 2 is a block diagram of components of the phacoemulsification system of FIG. 1 that drive and balance driving signals, together with a schematic side view of the phacoemulsification probe, in accordance with an example of the disclosure;

FIGS. 3A and 3B are, respectively, a graph of power levels of driving signals with the balancer disabled and enabled, and respectively resulting needle schematic trajectories, in accordance with an example of the disclosure;

FIG. 4 is an example of a schematic algorithm that the processor of the phacoemulsification system runs to vibrate the needle of the phacoemulsification probe in a circular trajectory, in accordance with an example of the disclosure; and

FIG. 5 is a flow chart that schematically illustrates a method for driving a needle of the phacoemulsification probe to vibrate in a circular trajectory, in accordance with an example of the disclosure.

### DETAILED DESCRIPTION OF EXAMPLES

### Overview

Phacoemulsification probes are commonly capable of multiple different modes of tip vibration, including, for example, longitudinal, transverse, torsional, and circular modes. For this purpose, it is desirable that the mechanism responsible for vibrating the distal tip of the probe be able to vibrate independently in three dimensions (3D). This sort of vibration can be implemented using a piezoelectric transducer in the probe.#

A transducer capable of 3D motion can be made, for example, from a single piezoelectric crystal, which is cut into two or more parts (e.g., angular segmented parts). These parts are cemented together, and a pair of electrodes is attached to each part. (Alternatively, two or more separate crystals can be cemented or otherwise attached together). This sort of device is referred to herein as a “multi-crystal.” The phases and amplitudes of the drive signals that are applied to each part of the multi-crystal are chosen to generate the desired mode of vibration.

Even when the multi-crystal is made by cutting a single crystal into parts, however, the different parts typically do not behave identically. For example, to vibrate an ideal multi-crystal in a circle, two pairs of electrodes could be energized with signals of the same frequency and voltage, but differing in phase by 90° so that the resultant vibrations of the parts of the multi-crystal are out of phase by 90°. In practice, the different parts of the multi-crystal have different electrical and mechanical characteristics, so that even applying equal voltages with a 90° phase difference may result in a vibration trajectory that is not circular, but rather elliptical. The voltages and the phase difference needed to generate a circular trajectory are not known a priori.

In more detail, the resulting multi-crystal no longer behaves exactly as a single crystal, due to, for example, limits on manufacturing capability, defects in the crystals, changes of temperature, and differences in the electrode plating. Therefore, while a tip driven by three angular segment parts of a crystal should theoretically vibrate in a circle if the driving signals to each segment are made equal except for phase differences of 0°, 120°, and 240°, in practice this is not the case.

Examples of the present disclosure that are described herein provide one or more controllers, collectively also termed a “balancer,” which controls driving signals outputted by a frequency generator for each of the number (e.g., three) of crystal segments. When operative, the balancer iteratively adjusts its output signals to the segments so that a selected parameter of the signals equalizes. In an example, individual processor-controlled balancer modules balance a selected signal between a given number of segment-driving channels.

In one example, the balancer is a software module on the digital path of the output signal chain. In another example, the balancer comprises physical hardware, which lets the balancer modify low-level signals (i.e., before the high-power amplifier/driver), as opposed to high-voltage signals (after the high-power amplifier/driver). However, in other examples, a balancer capable of adjusting high voltage signals can be placed at the output of a driver.

In one example, the selected signal parameter to balance between channels is the power level of the drive signals. In this case the voltages received from the frequency generator are varied by the balancer modules until the three output powers equalize. In another example, the selected signal parameter to balance between channels is current, in which case the output currents to the three segments are equalized.

Equalizing the output currents, for example by the balancer iteratively varying the input voltages, overcomes “misbehavior” of the multi-crystal. Thus, once the currents have been equalized, if the segments are driven with phase differences of 0°, 120°, and 240°, the tip vibrates in a circle.

Some of the disclosed examples further provide, irrespective of the balancer, individual processor-controlled drive modules to drive each segment at the vibration resonant-frequency. The different drive signal frequencies are adjusted independently of one another and enable vibration of the piezoelectric actuator continuously at the selected multimode resonant mode.

The balancer may be a standalone system that includes processing circuitry and software, or, alternatively, be an electrical unit controlled by an external processor, such as a processor of the phacoemulsification system.

Each of the separate drive modules and balancer modules may be realized in hardware or software, for example, in a proportional-integral-derivative (PID) control architecture.

### System Description

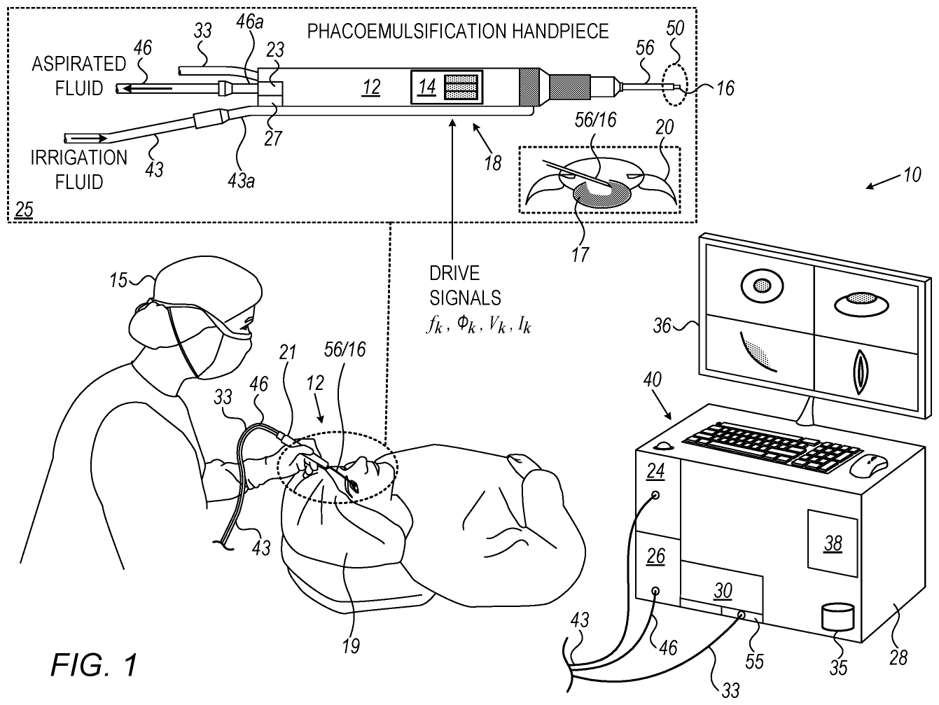

FIG. 1 is a schematic, pictorial view, along with a side view, of a phacoemulsification system 10, in accordance with an example of the present disclosure.

As seen in the pictorial view of phacoemulsification system 10, and in inset 25, phacoemulsification probe 12 (e.g., a handpiece 12) comprises a needle 16 surrounded by an irrigation sleeve 56. Needle 16 is hollow and its lumen is used as an aspiration channel. As seen, needle 16 can be moved in a circular trajectory 50.

Needle 16 is configured for insertion into a lens capsule 17 of an eye 20 of a patient 19 by a physician 15 to remove a cataract. The needle 16 (and irrigation sleeve 56) are shown in inset 25 as a straight object. However, any suitable needle may be used with phacoemulsification probe 12, for example, a curved or bent tip needle commercially available from Johnson & Johnson Surgical Vision, Inc., Irvine, Calif., USA.

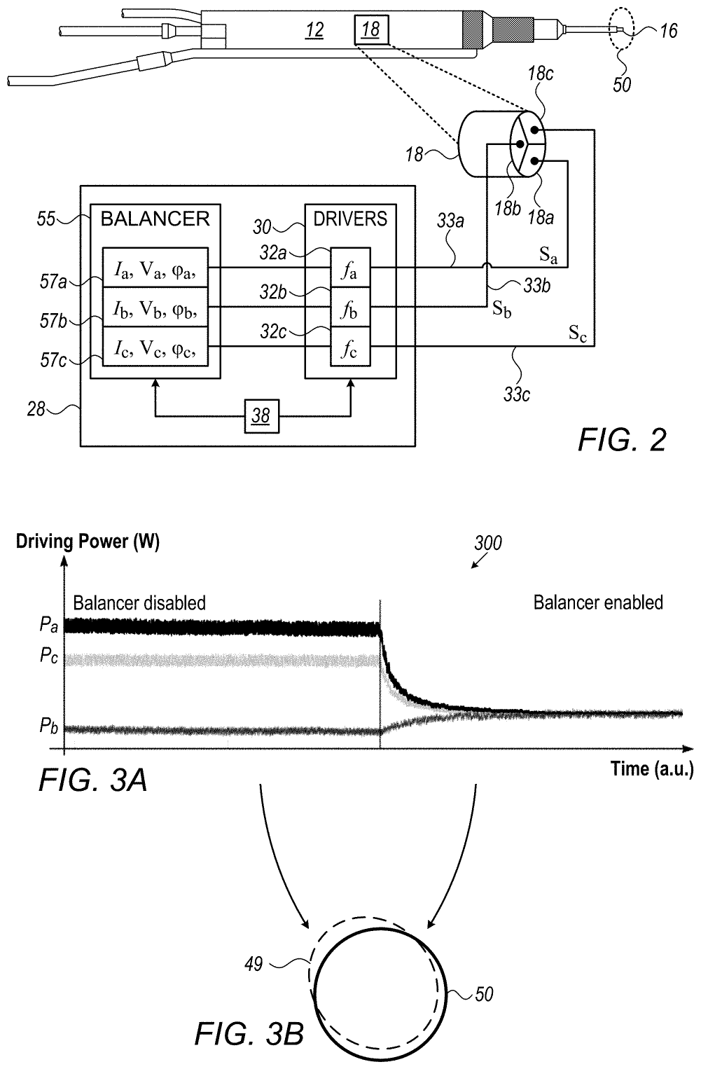

As further shown, phacoemulsification probe 12 includes a multi-crystal (shown in detail in FIG. 2) comprised in a piezoelectric actuator 14 coupled to a horn (not shown) that drives needle 16 to vibrate in the circular trajectory 50, that is used to break a cataract into small pieces during a phacoemulsification procedure. Console 28 comprises a piezoelectric drive module 30, coupled, via a balancer 55 described below, with the piezoelectric multi-crystal 18 using electrical wiring running in cable 33.

Drive module 30, which includes analog high-power filters/amplifiers/drivers (and has no control circuitry of its own in the shown example) is controlled by a processor 38 that uses the driving signals or small-amplitude monitoring signals (e.g., at detuned frequencies) via cable 33 and enables a multi-frequency mechanical resonance of multi-crystal 18 to be monitored and followed using balancer 55 adjusting driving signals.

Balancer 55 receives commands from processor 38, or runs an algorithm using an included processor, in order to adjust driving signals from drive module 30 for each of the number k (e.g., three) of crystal segments. When operative, typically anytime during which system 10 is turned on, the balancer iteratively adjusts some the drive signals (phases Φₖ, voltages Vₖ, currents Iₖ) that drive module 30 outputs to the segments so that a selected parameter of the signals equalizes. (Below, the phase terms Φₖ and φₖ are used interchangeably and mean the same thing.)

In one example, the selected parameter is power. In this case the voltages received from the frequency generator are varied until the three output powers equalize. If, in another example, the selected parameter is current, the output currents to the three segments are equalized (e.g., root mean square (RMS) values I₁=I₂=I₃). Typically, processor 38 separately controls the frequencies fₖ to maintain the different segmented parts so that each vibrates at its resonant frequency.

Equalizing the output currents, for example by iteratively varying the input voltages, overcomes “misbehavior” of the multi-crystal. Thus, once the currents have been equalized, if the segments are driven with phase differences of 0°, 120°, and 240°, the tip vibrates in a circle.

In the shown example, probe 12 includes a sensor 27 coupled with irrigation channel 43a, and a sensor 23 coupled with aspiration channel 46a. Channels 43a and 46a are coupled respectively to irrigation line 43 and aspiration line 46. The sensor measurements (e.g., pressure, vacuum, and/or flow) are taken close to the proximal end of the handpiece where the irrigation outlet and the aspiration inlet are located, so as to provide processor 38 an accurate indication of the actual measurements occurring within an eye and provide a short response time to a control loop comprised in processor 38.

As shown, during the phacoemulsification procedure, processor-controlled pump 24 comprised in a console 28 pumps irrigation fluid from an irrigation reservoir (not shown) via irrigation sleeve 56 to irrigate the eye. The fluid is pumped via irrigation tubing line 43 running from console 28 to probe 12. Using sensors (e.g., as indicated by sensors 23 and/or 27), processor 38 controls a pump rate of irrigation pump 24 to maintain intraocular pressure within prespecified limits.

Eye fluid and waste matter (e.g., emulsified parts of the cataract) are aspirated via hollow needle 16 to a collection receptacle (not shown) by a processor-controlled aspiration pump 26 also comprised in console 28 and using aspiration tubing line 46 running from probe 12 to console 28. In an example, processor 38 controls an aspiration rate of aspiration pump 26 to maintain intraocular pressure (in case of sub-pressure indicated, for example, by sensor 23) within prespecified limits.

In the shown example, processor 38 may receive user-based commands via a user interface 40, which may include setting a vibration mode, and setting or adjusting an irrigation and/or aspiration rate of the irrigation pump 24 and aspiration pump 26. Processor 38 may receive user-based commands via a user interface 40, which may include needle 16 stroke amplitude settings and turning on irrigation and/or aspiration.

In an example, the physician uses a foot pedal (not shown) as a means of control. For example, pedal position one activates only irrigation, pedal position two activates both irrigation and aspiration, and pedal position three adds needle 16 vibration. Additionally, or alternatively, processor 38 may receive the user-based commands from controls located in a handle 21 of probe 12.

In an example, user interface 40 and display 36 may be integrated into a touch screen graphical user interface.

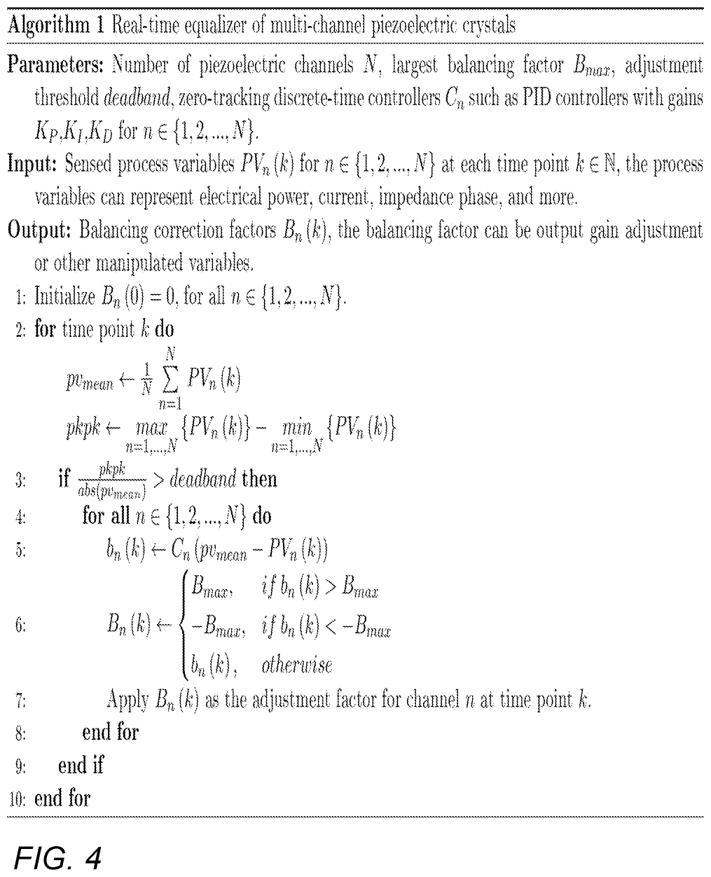

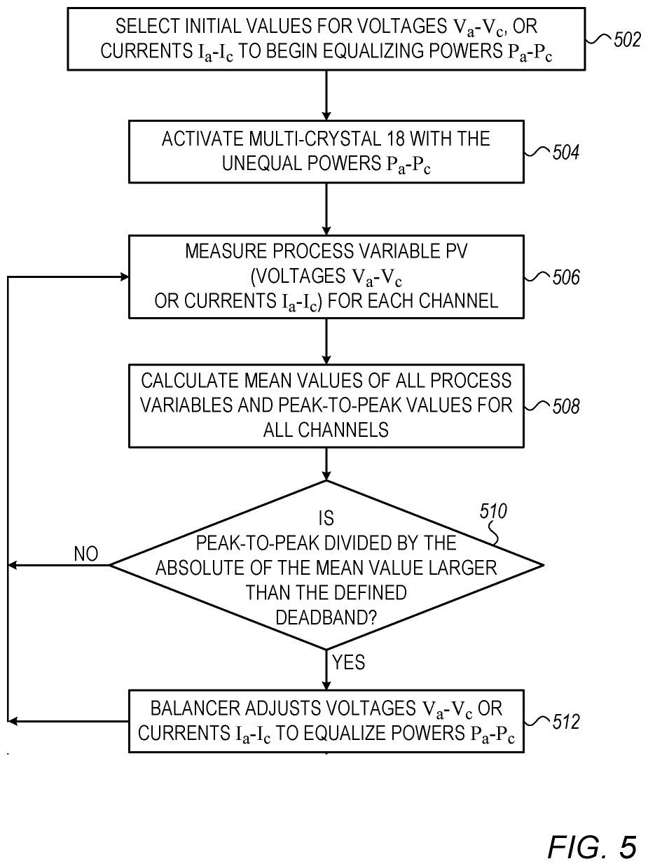

Some or all of the functions of processor 38 may be combined in a single physical component or, alternatively, implemented using multiple physical components. These physical components may comprise hard-wired or programmable devices, or a combination of the two. In some examples, at least some of the functions of processor 38 may be carried out by suitable software stored in a memory 35 (as shown in FIG. 1). This software may be downloaded to a device in electronic form, over a network, for example. Alternatively, or additionally, the software may be stored in tangible, non-transitory computer-readable storage media, such as optical, magnetic, or electronic memory. In particular, processor 38 runs a dedicated algorithm as disclosed herein, included in FIGS. 4 and 5, that enables processor 38 to perform the disclosed steps, as further described below.

The system shown in FIG. 1 may include further elements which are omitted for clarity of presentation. For example, physician 15 typically performs the procedure using a stereo microscope or magnifying glasses, neither of which are shown. Physician 15 may use other surgical tools in addition to probe 12, which are also not shown in order to maintain clarity and simplicity of presentation.#

### Equalized Multi-Channel Driving of Segmented Piezoelectric Crystals

FIG. 2 is a block diagram of components (30, 38, 55) of the phacoemulsification system 10 of FIG. 1 that drive and balance driving signals, together with a schematic side view of the phacoemulsification probe 12, in accordance with an example of the disclosure.

As seen, multi-crystal 18 drives the vibration of tip 16 in response to drive signals applied by processor 38-controlled drive module 30. In an example, multi-crystal 18 may comprise three parts, i.e., three piezoelectric crystals 18a, 18b, and 18c that are coupled, e.g., cemented, together. Drive module 30 comprises three driver circuits 32a, 32b, and 32c. Driver circuits 32a-32c are coupled respectively with piezoelectric crystals 18a-18c via respective wires 33a, 33b and 33c of cable 33, and actuate the crystals with respective harmonic signals Sₐ, S_{b}, and S_{c}, having respective frequencies fₐ, f_{b}, and f_{c}.

Harmonic signals Sₐ, S_{b}, and S_{c} can be characterized by voltages Vₐ, V_{b}, and V_{c}, currents Iₐ, I_{b}, and I_{c} and phases φₐ, φ_{b}, and φ_{c}. Given that the three piezoelectric crystals 18a, 18b, and 18c each exhibit frequency-dependent impedance (Zₐ, Z_{b}, and Z_{c}), the relationship between voltages, currents, and phases of driving signal are complicated.

As noted above, a balancer 55 receives commands from an external processor, or runs an algorithm using an included processor, so as to adjust driving signals from driving module 30 for the three crystal segments. When operative, the balancer iteratively adjusts the signals it outputs to the segments so that a selected parameter of the signals equalizes.

To this end, in the shown example, balancer 55 comprises three balancer modules 57a, 57b, and 57c, that may be realized in hardware or software, for example, in a proportional-integral-derivative (PID) control architecture. In the shown example, balancer modules 57a, 57b, and 57c are commanded by processor 38 that runs a dedicated algorithm, such as described in FIG. 4.

In one example, the selected parameter is power. In this case the voltages received from the frequency generator are varied until the three output powers Pₐ, P_{b}, and P_{c} equalize. If, in another example, the selected parameter is current, then the output currents to the three segments are equalized.

Equalizing the output currents, for example by iteratively varying the voltages Vₐ, V_{b}, and V_{c}, overcomes “misbehavior” of the multi-crystal. Thus, once the currents have been equalized, if the segments are driven with phase differences of 0°, 120°, and 240°, the tip vibrates in a circle.

Balancer 55 may comprise analog and/or digital circuits and interfaces enabling it to carry out the functions described herein.

The example block diagram shown in FIG. 2 is chosen purely for the sake of conceptual clarity. In alternative examples, balancer 55 is a standalone unit that includes a memory and a processor to store and run an algorithm such as described in FIG. 4.

FIGS. 3A and 3B are, respectively, a graph 300 of driving signal power with balancer 55 disabled and enabled, and respectively resulting needle schematic trajectories 49 and 50, in accordance with an example of the disclosure.

As seen, while balancer 55 is disabled, the three output powers Pₐ, P_{b}, and P_{c} are unequal, with Pₐ>P_{c}>P_{b}. At this point needle 16 vibrates with a clinically suboptimal elliptical trajectory 49. A given time duration after balancer 55 is enabled, the balancer equalizes the powers, Pₐ=P_{c}=P_{b}. The time duration for balancer 55 to equalize the powers is typically up to several tens of milliseconds. When driving powers Pₐ, P_{b}, and P_{c} are equal, needle 16 vibrates with the clinically optimal circular trajectory 50.

The example trajectories shown in FIG. 3B are chosen purely for the sake of conceptual clarity. In alternative examples, depending on the arrangement of the piezoelectric crystals, balancer 55 can iteratively adjust the signals so that a selected function of the signal parameters can be reached, such as a ratio of powers, so as to cause the tip to vibrate at another predefined trajectory in three dimensions.

FIG. 4 is an example of a schematic algorithm that the processor 38 of the phacoemulsification system 10 runs to vibrate needle 16 of phacoemulsification probe 12 in circular trajectory 50, in accordance with an example of the disclosure. The given example of the balancing algorithm is called “Algorithm 1.”

As seen, Algorithm 1 is coded for use with a number N of PID controllers to drive N piezoelectric segments. In FIG. 2, N=3. The algorithm receives “n” as it is termed in Algorithm 1, as an input parameter of the signal to be sensed and equalized. For example, “n” may be the power P. The algorithm includes a step of comparing a ratio of the peak-to-peak value of each process variable (PV) channel and an absolute value of the mean value of PV to a preset threshold (e.g., to 1), the threshold also called “deadband”. A PV can be, for example, the different driving voltages Vₐ, V_{b}, and V_{c} or the currents Iₐ, I_{b}, and I_{c}.

Algorithm 1 is a closed loop control code that, for each of the PID controllers (e.g., for modules 57a, 57b and 57c of FIG. 2), iteratively receives measured respective powers Pₐ, P_{b}, and P_{c} and acts (by lines 3-9 of Algorithm 1) to equalize them by updating adjustment factors Bₙ(k), with k being an iteration step (k being a time point).

The example the schematic high-level algorithm (e.g., a pseudo-code) shown in FIG. 4 is chosen purely for the sake of conceptual clarity. In practice a more detailed algorithm may be used.

FIG. 5 is a flow chart that schematically illustrates a method for driving a needle of the phacoemulsification probe to vibrate in a circular trajectory, in accordance with an example of the disclosure. The algorithm, according to the presented example, carries out a process that begins with processor 38 selecting initial voltages Vₐ-V_{c} or currents Iₐ-I_{c}, and phases φₐ-φₘ for signals Sₐ-S_{c} (e.g., powers Pₐ-P_{c}), in an initial driving signals selection step 502.

In an activation step 504, processor 38 activates (by controlling drive module 30) multi-crystal 18 with the unequal powers Pₐ-P_{c}, which result is a certain clinically suboptimal trajectory, such as elliptical trajectory 49.

Next, by running the algorithm, such as Algorithm 1 of FIG. 4, balancer 55 measures process variable PV (e.g., voltage or current) for each channel, at a process variables measuring step 506.

Next, processor 38 (or balancer 55) calculates a mean value of the process variables and a peak-to-peak value of each process variable, at a process variables calculation step 508.

At a checking step 510, the processor checks if a peak-to-peak value of a process variable of any channel is larger than the mean value, by checking if a ratio of peak-to-peak value of each process variable (PV) channel and an absolute value of the mean value of PV to a preset deadband value.

If the answer us “No,” the process returns to step 506.

If the answer is “Yes,” processor 38 controls separate modules 57a, 57b, and 57c of balancer 55, to adjust voltages Vₐ-V_{c}, or currents Iₐ-I_{c}, so as to equalize powers Pₐ-P_{c}, at a balancing step 512.

The equalized powers Pₐ-P_{c} result in a clinically optimal trajectory, such as circular trajectory 50.

The example flow chart shown in FIG. 5 is chosen purely for the sake of conceptual clarity. In alternative examples, balancer 55 is a standalone unit that includes a memory and one or more processors to perform the equalization without need in using external processor 38.

### Example 1

A method for driving a medical probe (12), the method including applying respective harmonic electrical signals (Sa, Sb, Sc) to multiple piezoelectric crystals (18a, 18b, 18c) coupled with a tip (16) of the medical probe (12) so as to cause the tip to vibrate. The signals are iteratively adjusted to equalize a selected parameter of the signals, so as to cause the tip to vibrate at a predefined trajectory.

### Example 2

The method according to claim 1, wherein applying the respective harmonic electrical signals (Sa, Sb, Sc) comprises applying the signals at respective resonant frequencies of the multiple piezoelectric crystals (18a, 18b, 18c).

### Example 3

The method according to claim 1, wherein the selected parameter is driving power, and wherein iteratively adjusting the signals comprises adjusting one of driving voltages and driving currents of the signals.

### Example 4

The method according to claim 1, wherein the selected parameter is driving current, and wherein iteratively adjusting the signals comprises adjusting one of driving voltages and phases of the signals.

### Example 5

The method according to claim 1, wherein the multiple piezoelectric crystals (18a, 18b, 18c) are shaped as angular segments, and wherein the predefined trajectory is circular (50).

### Example 6

The method according to claim 1, wherein the tip comprises a needle (16) of a phacoemulsification probe (12), the needle (16) inserted into a lens capsule of an eye (20), and being vibrated so as to emulsify a cataracted lens (17).

### Example 7

A system for driving a medical probe, the system comprising:

a processor (38), which is configured to apply respective harmonic electrical signals (Sa, Sb, Sc) to multiple piezoelectric crystals (18a, 18b, 18c) coupled with a tip of the medical probe (12) so as to cause the tip to vibrate; and

a balancer (55), which is configured to iteratively adjust the signals (Sa, Sb, Sc) to equalize a selected parameter of the signals (Sa, Sb, Sc), so as to cause the tip to vibrate at a predefined trajectory.

Although the examples described herein mainly address Phacoemulsification systems, the methods and systems described herein can also be used in other applications, such as balancing speakers output to balance audio level in a specific position in a room, or balancing power of several light sources from different positions to equalize lighting on an object from several directions at once.

It will thus be appreciated that the examples described above are cited by way of example, and that the present disclosure is not limited to what has been particularly shown and described hereinabove. Rather, the scope of the present disclosure includes both combinations and subcombinations of the various features described hereinabove, as well as variations and modifications thereof which would occur to persons skilled in the art upon reading the foregoing description and which are not disclosed in the prior art.

1. A method for driving a medical probe, the method comprising:
applying respective harmonic electrical signals to two or more piezoelectric crystals coupled with a tip of the medical probe so as to cause the tip to vibrate; and
iteratively adjusting the signals to equalize a selected parameter of the signals, so as to cause the tip to vibrate at a predefined trajectory.

2. The method according to claim 1, wherein applying the respective harmonic electrical signals comprises applying the signals at respective resonant frequencies of the two or more piezoelectric crystals.

3. The method according to claim 1, wherein the selected parameter is driving power, and wherein iteratively adjusting the signals comprises adjusting one of driving voltages and driving currents of the signals.

4. The method according to claim 1, wherein the selected parameter is driving current, and wherein iteratively adjusting the signals comprises adjusting one of driving voltages and phases of the signals.

5. The method according to claim 1, wherein the two or more piezoelectric crystals are shaped as angular segments, and wherein the predefined trajectory is circular.

6. The method according to claim 1, wherein the tip comprises a needle of a phacoemulsification probe, the needle inserted into a lens capsule of an eye, and being vibrated so as to emulsify a cataracted lens.

7. A system for driving a medical probe, the system comprising:
a processor, which is configured to apply respective harmonic electrical signals to two or more piezoelectric crystals coupled with a tip of the medical probe so as to cause the tip to vibrate; and
a balancer, which is configured to iteratively adjust the signals to equalize a selected parameter of the signals, so as to cause the tip to vibrate at a predefined trajectory.

8. The system according to claim 7, wherein the processor is configured to apply the harmonic electrical signals at respective resonant frequencies of the two or more piezoelectric crystals.

9. The system according to claim 7, wherein the selected parameter is driving power, and wherein the balancer is configured to iteratively adjust the signals by adjusting one of driving voltages and driving currents of the signals.

10. The system according to claim 7, wherein the selected parameter is driving current, and wherein the balancer is configured to iteratively adjust the signals by adjusting one of driving voltages and phases of the signals.

11. The system according to claim 7, wherein the two or more piezoelectric crystals are shaped as angular segments, and wherein the predefined trajectory is circular.

12. The system according to claim 7, wherein the tip comprises a needle of a phacoemulsification probe, the needle inserted into a lens capsule of an eye, and being vibrated so as to emulsify a cataracted lens.
